(19) United States
(12) Patent Application Publication
(10) Pub. No.: US 2026/0151172 A1
(43) Pub. Date: Jun. 4, 2026

(54) THROMBUS DEBULKING DEVICES, SYSTEMS, AND METHODS

(71) Applicant: Boston Scientific Scimed, Inc., Maple Grove, MN (US)

(72) Inventors: Cass Alexander Hanson, St. Paul, MN (US); Bruce R. Forsyth, Hanover, MN (US); Tina Marie Lenneman, Otsego, MN (US); Timothy A. Ostroot, Cokato, MN (US)

(73) Assignee: Boston Scientific Scimed, Inc., Maple Grove, MN (US)

(21) Appl. No.: 19/404,687

(22) Filed: Dec. 1, 2025

Related U.S. Application Data
(60) Provisional application No. 63/726,588, filed on Dec. 1, 2024.

(51) Int. Cl. A61B 18/00

(52) U.S. Cl. CPC A61B 18/00; A61B 2018/00083; A61B 2018/0022; A61B 2018/0041; A61B 2218/007

(57) ABSTRACT

A pulse field ablation system may include a first elongate shaft, a second elongate shaft, one or more electrodes, a wave generator, and a treatment control module. The first elongate shaft may include one or more lumens with a first lumen configured to pass a fluid to treat a thrombus in a vessel of a subject. The second elongate shaft may be configured to extend in a distal direction beyond a distal end of the first shaft. The one or more electrodes may be disposed at a distal portion of the second elongate shaft. The wave generator may be configured to be in electrical communication with the one or more electrodes. The treatment control module may selectively cause the wave generator to deliver energy to an electrode of the one or more electrodes, where the energy may be configured to lyse red blood cells of the thrombus.

### CROSS-REFERENCE TO RELATED APPLICATIONS

This application claims the benefit of priority under 35 U.S.C. §119 of U.S. Provisional Application No. 63/726,588, filed December 1, 2024, the entire disclosure of which is hereby incorporated by reference.

### TECHNICAL FIELD

The present disclosure pertains to thrombus treatment devices, systems, and methods. More specifically, the present disclosure relates to pulse field ablation devices, systems, and methods configured to treat thrombus in vasculature of a subject.

### BACKGROUND

A wide variety of intracorporeal and extracorporeal medical devices and systems have been developed for medical use, for example, in vasculature procedures and/or for vasculature treatments. Some of these devices and systems include guidewires, catheters, catheter systems, pump devices, ablation device, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including pulse field ablation devices for treating a thrombus in vasculature of a subject.

In a first example, a system may include a medical device comprising an elongate shaft and one or more electrodes disposed at a distal portion of the elongate shaft, a wave generator configured to be in electrical communication with the one or more electrodes, and a controller configured to selectively cause the wave generator to deliver pulse field ablation energy to an electrode of the one or more electrodes, the pulse field ablation energy is configured to lyse red blood cells of a thrombus in a vessel of a subject.

Alternatively or additionally to any of the examples above, the elongate shaft may be a second elongate shaft and the medical device may include a first elongate shaft having a lumen configured to pass fluid to treat a thrombus in a vessel of a subject.

Alternatively or additionally to any of the examples above, the system may further include an aspiration source configured to be in fluid communication with a lumen of the medical device to remove fibrin of the thrombus from the vessel of the subject in response to actuation of the aspiration source.

Alternatively or additionally to any of the examples above, the system may further include a source of a thrombolytic drug configured to be in fluid communication with a lumen of the medical device to lyse fibrin of the thrombus in the vessel of the subject with the thrombolytic drug in response to actuation of the source of the thrombolytic drug.

Alternatively or additionally to any of the examples above, the one or more electrodes may include at least two electrodes in a bipolar arrangement along the elongate shaft.

Alternatively or additionally to any of the examples above, the one or more electrodes may include an electrode in a monopolar arrangement along the elongate shaft.

Alternatively or additionally to any of the examples above, the system may further include a grounding pad configured to be located external of the subject and create an electric field with the electrode in the monopolar arrangement along the elongate shaft.

Alternatively or additionally to any of the examples above, the elongate shaft may include an expandable member at the distal portion and the one or more electrodes are disposed at a longitudinal location of the expandable member.

Alternatively or additionally to any of the examples above, the medical device may further comprises a lumen configured to facilitate radial adjustment of the expandable member.

Alternatively or additionally to any of the examples above, the system may further include an inflation fluid source fluidly coupled with the lumen of the medical device, wherein the expandable member may be a balloon configured to receive inflation fluid from the inflation fluid source via the lumen of the elongate shaft.

Alternatively or additionally to any of the examples above, the system may further include an insulative sleeve configured to be adjustably disposed over a portion of the elongate shaft to control a location of an electric field from the one or more electrodes.

In another example, a medical device may include a first elongate shaft comprising a first lumen, the first elongate shaft is configured to pass through a vessel of a subject and the first lumen is configured to pass fluid to treat a thrombus in the vessel of the subject, a second elongate shaft configured to extend in a distal direction from a distal end of the first elongate shaft, and one or more electrodes disposed at a distal portion of the second elongate shaft, wherein the one or more electrodes may be configured to apply pulse field ablation (PFA) energy to the thrombus to lyse red blood cells of the thrombus.

Alternatively or additionally to any of the examples above, the distal portion of the second elongate shaft may include an expandable member and the one or more electrodes are disposed along a longitudinal location of the expandable member.

Alternatively or additionally to any of the examples above, the one or more electrodes include a plurality of electrodes configured in a bipolar arrangement.

Alternatively or additionally to any of the examples above, the device may further include an insulative sleeve disposed on a portion of the second elongate shaft to control a degree of electrode contact and length of longitudinal treatment using the one or more electrodes.

In another example, a method for treating a thrombus may include advancing a catheter system to a treatment site in a vessel containing a thrombus, the catheter system comprising an elongate shaft with one or more electrodes disposed along a distal portion of the elongate shaft, positioning the one or more electrodes at the thrombus, generating an electric field at the one or more electrodes to deliver pulse field ablation (PFA) energy to red blood cells of the thrombus and cause irreversible electroporation of the red blood cells, and treating fibrin of the thrombus after the PFA energy is delivered to the red blood cells of the thrombus.

Alternatively or additionally to any of the examples above, the method may further include delivering a conductive fluid to the treatment site prior to delivering the PFA energy to the red blood cells of the thrombus.

Alternatively or additionally to any of the examples above, the elongate shaft may include an expandable member and the one or more electrodes are positioned at a longitudinal location of the expandable member and positioning the one or more electrodes at the thrombus may include radially expanding the expandable member.

Alternatively or additionally to any of the examples above, treating the fibrin of the thrombus after the PFA energy is delivered to the red blood cells of the thrombus may include aspirating the fibrin from the vessel.

Alternatively or additionally to any of the examples above, treating the fibrin of the thrombus after the PFA energy is delivered to the red blood cells of the thrombus may include applying a thrombolytic drug to the fibrin to lyse the fibrin.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative configuration of a pulse field ablation system;

FIG. 2 is a schematic diagram of an illustrative configuration of a controller/control module and a user interface;

FIG. 3A is a schematic side view of an illustrative configuration of a pulse field ablation system;

FIG. 3B is a schematic cross-section view of the pulse field ablation system depicted in FIG. 3A, taken along line 3B-3B;

FIG. 4 is a schematic side view of an illustrative configuration of a pulse field ablation system;

FIG. 5 is a schematic side view of an illustrative configuration of a pulse field ablation system;

FIG. 6A is a schematic side view of an illustrative configuration of a pulse field ablation system in a first position;

FIG. 6B is a schematic cross-section view of the pulse field ablation system depicted in FIG. 6A, taken along line 6B-6B;

FIG. 6C is a schematic cross-section view of the pulse field ablation system depicted in FIG. 6A in a second position;

FIGS. 7A and 7B are side views of an illustrative configuration of a pulse field ablation system in a first position and a second position, respectively;

FIGS. 8A and 8B are side views of an illustrative configuration of a pulse field ablation system in a first position and a second position, respectively;

FIG. 9 is a schematic side view of an illustrative configuration of electrodes on a portion of a pulse field ablation device;

FIG. 10 is a schematic side view of an illustrative configuration of electrodes on a portion of a pulse field ablation device;

FIG. 11 is a schematic end view of an illustrative configuration of electrodes on a portion of a pulse field ablation device;

FIG. 12 is a schematic diagram of an illustrative technique for using a pulse field ablation system for treating a thrombus in vasculature of a subject; and

FIGS. 13A-13D are schematic diagrams of an illustrative technique for using a pulse field ablation system for treating a thrombus in vasculature of a subject.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Electroporation is the generation of a destabilizing electric potential across a biological cell membrane. Destabilizing electric potentials that induce electroporation are generally in the range of several hundred volts across a distance of several millimeters. In electroporation, pores are formed when a voltage across the biological cell membrane exceeds a dielectric strength of the biological cell membrane. In some examples, the pores created by applying electrical potentials across a biological cell membrane may facilitate inducing a substance into a cell, such as loading the cell with a drug, a piece of coding DNA, etc.

If the strength of an electric field applied to a cell and/or duration of exposure by the cell to the electric field are suitably chosen to not reach a threshold, the pores formed in the cell by the electrical pulse will reseal after a short period of time. Such a process is called reversible electroporation (RE) and does not permanently damage a membrane of the cell. If the strength of the electric field to the cell and/or duration of exposure of the cell to the electric field are suitably chosen to reach or exceed the threshold, the applied electric field across cell membranes can cause apoptosis and/or necrosis resulting in cell death. Such a process is called irreversible electroporation (IRE). The electric field that results in RE or IRE may be applied via pulse field ablation and/or other suitable techniques.

Pulse field ablation (PFA) therapy is configured to isolate and target specific tissues for IRE to induce cell death using a non-thermal or low-thermal energy source. Unlike thermal ablation methods, PFA may be used to target specific tissues or specific cell type, which may mitigate risk of collateral injury to tissue or cells adjacent to the targeted tissues or cells. As discussed herein, IRE inducing PFA may be used to treat a thrombus in vasculature of a subject (e.g., a patient and/or other suitable subject).

The two main constituents of acute and subacute thrombus in vasculature of a subject are red blood cells (RBCs) and fibrin. In some cases, an aspiration device may be used to debulk a thrombus from the vasculature of a subject by removing fibrin and RBCs using a force caused by a pressure differential. Alternatively or additionally, a lytic may be used to debulk the thrombus by lysing the fibrin of the thrombus so that the RBCs are released and passed through the vasculature. These techniques, although effective in debulking a thrombus in vasculature of a subject, may result in damage to tissue around the thrombus and/or cause other injury to the subject.

The present disclosure provides medical devices, systems, and methods configured to debulk a thrombus in vasculature of a subject by lysing RBCs through the use of PFA energy that induces IRE of the RBCs. As RBCs may form a majority of a thrombus, lysing the RBCs is an effective way to reduce a solid volume of the thrombus and treat the thrombus. In some examples, an intravascular based catheter system (e.g., a PFA system) may be configured to propagate a specific microsecond electrical waveform to create irreversible electrical disruption of RBC cellular membranes dispersed through a fibrin matrix of a thrombus to lyse the RBCs. The catheter system may include an aspiration feature that allows for removal of the fibrin matrix after the RBCs have been lysed. Alternatively or additionally, the catheter system may include a thrombolytic feature used to infuse a thrombolytic drug to lyse the remaining fibrin matrix after the RBCs have been lysed.

The catheter system may include one or more electrodes located at a distal portion of the catheter system that is configured to be positioned in or proximate a thrombus in the vasculature of the subject. The one or more electrodes and/or components of the catheter system may have any suitable configuration to allow for shaping an electric field therewith and/or controlling a depth of the electric field across the thrombus. In some examples, the catheter system may include an insulative sleeve configured to control a degree of surface electrode contact and/or length of the pulsed-electric field therapy. In some examples, the catheter system may be configured to facilitate applying a fluid to the thrombus area to induce or weaken conductivity at the thrombus prior to or during the PFA therapy.

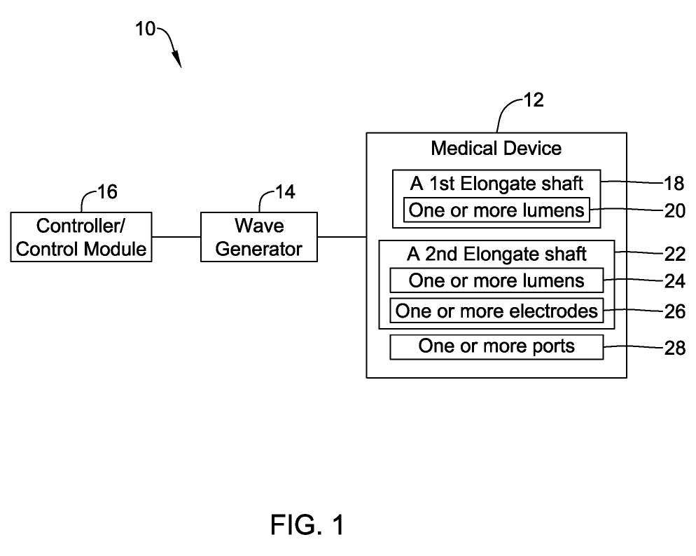

FIG. 1 is a schematic diagram of an illustrative configuration of a pulse field ablation (PFA) system 10 (e.g., a catheter system and/or other suitable type of system). The system 10 may include any suitable components for performing PFA therapy or treatments on a thrombus in a vasculature of a subject (e.g., a patient). For example, the system 10 may include, among other suitable components, a medical device 12 (e.g., a catheter, etc.), a wave generator 14, and a controller/control module 16 (hereinafter “controller”). The wave generator 14 and the controller 16 may be a single component or separate components in communication with one another. The medical device 12 may include, among other suitable components, a first elongate shaft 18 having one or more lumens 20, a second elongate shaft 22 having one or more lumens 24 and one or more electrodes 26, and one or more ports 28. The one or more electrodes 26 may be in electrical communication with the wave generator 14 and/or the controller 16 via wires or leads extending through the one or ports 28. Although the one or more electrodes 26 are depicted in FIG. 1 as being part of the second elongate shaft 22, one or more of the electrodes 26 may be part of or located on the first elongate shaft 22 and/or other suitable component of the system 10.

The first elongate shaft 18 may have any suitable configuration. In some examples, the first elongate shaft 18 may be configured to extend from a hub of the system 10 located exterior of the vasculature of the subject to a location proximate the thrombus in the vasculature of the subject. The one or more lumens 20 of the first elongate shaft 18 may be configured to extend along at least a portion of a length of the first elongate shaft 18. In some examples, at least one of the one or more lumens 20 of the first elongate shaft 18 may be coupled with and/or in fluid communication with at least one of the one or more ports 28.

The one or more lumens 20 of the first elongate shaft 18 may have any suitable configuration and/or purpose. For example, the one or more lumens 20 of the first elongate shaft 18 may be configured to receive inflation fluid, aspiration pressure, one or more expansion member shafts, one or more control wires, one or more electrical wires, a guidewire, and/or may be configured to receive or facilitate other suitable features of the system 10. In one example, the first elongate shaft 18 may include a guidewire lumen, an electrical wire or connector lumen, an aspiration lumen, and an expansion source lumen (e.g., an inflation lumen, an expansion member shaft lumen, etc.). Other suitable configurations of the one or more lumens 20 of the first elongate shaft 18 are contemplated.

The second elongate shaft 22 may have any suitable configuration. The second elongate shaft 22 may be part of the first elongate shaft 18 and/or may be separate from the first elongate shaft 18. In some examples, the second elongate shaft 22 may be configured to extend from a hub of the system 10 to a location distal of a distal end of the first elongate shaft 18. Alternatively, the second elongate shaft 22 may be configured to extend from a location proximal of or distal of the hub of the system 10 to a location distal of the distal end of the first elongate shaft 18. The second elongate shaft 22 may be fixed relative to the first elongate shaft 18 or the second elongate shaft 22 may be longitudinally and/or rotationally adjustable relative to first elongate shaft 18. When the second elongate shaft 22 is longitudinally and/or rotationally adjustable relative to the first elongate shaft 18, a control mechanism (e.g., an actuator and/or other suitable control mechanism) may be located at or proximate the hub or may be part of the hub of the system 10 and longitudinally and/or rotationally adjustable relative to a housing of the hub to longitudinally and/or rotationally adjust one or both of the second elongate shaft 22 and the first elongate shaft 18 relative to the other. The control mechanism may be or may include one or more control components (e.g., a control button, a control wheel, a control slide, a shaft extending from the second elongate shaft 22 to the hub (e.g., in communication with the control button) and/or through a port of the hub).

The first elongate shaft 18 and/or the second elongate shaft 22 may include or may be coupled with one or more expandable members at a distal end or portion thereof. The one or more expandable members may have any suitable configuration including, but not limited to, expandable structures, self-expandable structures, balloon expandable structures, a balloon, an expandable cage, expandable strands or struts, and/or other suitable configurations. In some examples, the one or more expandable members may include one or more balloons configured to expand in response to fluid being delivered to the balloons via an inflation lumen. In some examples, the one or more expandable members may include an expandable structure configured to expand in response to distally or proximally extending a shaft or sleeve relative to the expandable member.

The one or more expandable members may have any suitable shape. For example, the shape of the one or more expandable members may be or may include an elongate shape, a cylindrical shape, a bulbous shape, a sinusoidal shape, and/or any other suitable shape. In some examples, the one or more expandable members may extend longitudinally along and circumferentially around a central longitudinal axis of the medical device 12. In one example configuration of an expandable member, the expandable member may be shaped so as to extend circumferentially around a guidewire lumen and/or other suitable lumen and define one or more lobes.

The one or more expandable members may be formed from any suitable material. For example, the one or more expandable members may be formed from materials including, but not limited to, non-compliant materials, semi-compliant materials, polymers, metals, alloys, shape memory metals, shape memory polymers, polyamides, polyimides polyesters, polyethers, polyacrylates, nylons, polyethylene terephthalate (PET), polyurethanes, polyolefins (e.g., HDPE, etc.), and/or other suitable materials.

The one or more lumens 24 of the second elongate shaft 22 may have any suitable configuration and/or purpose. For example, the one or more lumens 24 of the second elongate shaft 22 may be configured to receive inflation fluid, aspiration pressure, one or more expansion member shafts, one or more control wires, one or more electrical wires, a guidewire, and/or may be configured to receive or facilitate receiving other suitable features of the system 10. In one example, the second elongate shaft 22 may include a guidewire lumen and an expansion source lumen (e.g., an inflation lumen, an expansion member shaft lumen, etc.) Other suitable configurations of the one or more lumens 24 of the second elongate shaft 22 are contemplated.

The one or more electrodes 26 may have any suitable configuration. For example, the one or more electrodes 26 may include one or more ring electrodes 26 extending circumferentially around a shaft, one or more segmented radial ring electrodes extending circumferentially around a shaft, one or more elongate electrodes 26 extending along a length of a shaft, one or more electrodes extending at least partially around a circumference of a shaft, one or more flexible electrodes, and/or other suitable types of electrodes. The one or more electrodes 26 may include one or more pairs of electrodes in a bipolar arrangement or configuration with respect to one another. When the one or more electrodes 26 include a plurality of pairs of electrodes 26, an electrode 26 from one pair of electrodes 26 may be an electrode 26 in another pair of electrodes 26 or both electrodes 26 from an electrode pair may be separate or independent from electrodes 26 of another electrode pair. Alternatively or additionally, the one or more electrodes 26 may include an electrode 26 having a monopolar arrangement or configuration that is in electrical communication with a grounding pad exterior of the subject. When the second elongate shaft 22 includes an expandable portion, the one or more electrodes 26 may be located on or along the expandable portion to ensure good electrical contact with intended target cells of the subject, but other suitable configurations are contemplated.

Any suitable number of electrodes 26 may be positioned along the first elongate shaft 18 and/or the second elongate shaft 22. When a plurality of electrodes 26 and/or electrode pairs are located along the second elongate shaft 22, the electrodes 26 or electrode pairs may be triggered or fired in series or in parallel. In one example, the electrode 26 may be fired in parallel to provide individual control of one or more of the electrodes 26 or pairs of electrodes. In some examples, the electrodes 26 and/or electrode pairs may be controlled via multiplexing or with a multiplexer.

The one or more electrodes 26 may have any suitable configuration on the first elongate shaft 18 and/or the second elongate shaft 22. For example, the one or more electrodes 26 may be arranged on the second elongate shaft 22 to create one or more distinct electric fields, create a circumferentially asymmetrical electric field, create a circumferentially symmetrical electric field, create a radially outward electric field, create a forward-facing electric field, create a particular depth of electric field (e.g., an electric field extending a desired radial and/or longitudinal distance from the electrode), and/or create one or more other suitable electric field configurations. In some examples, when two spaced apart electrodes 26 of an electrode pair are closer together, the electric field may extend farther radially outward from the electrodes than when the electrodes are spaced farther apart, assuming all other parameters (e.g., electric field, impedance, voltage, waveform, etc.) remain the same. Other suitable configurations of the electrodes 26 are contemplated.

The wave generator 14 may be configured to generate electrical pulses to the electrodes 26, where the electrical pulses are configured to cause IRE of RBCs proximate the electrodes 26. In some examples, the wave generator 14 may be a voltage pulse waveform generator and provide a voltage pulse waveform to the electrodes 26 for performing PFA.

The wave generator 14 may be configured to generate and/or deliver any suitable type of signal for or to the electrodes 26. Example suitable types of signals generated by the wave generator 14 include, but are not limited to, , direct current (DC) pulses (such as high-voltage, ultra-short pulses used in electroporation), stimulus range pulses, and/or hybrid electrical pulses. In one example, the wave generator 14 may generate monophasic (DC) pulses and biphasic (DC) pulses. It is contemplated that the wave generator 14 may be configured to provide other suitable types of signals to the electrodes.

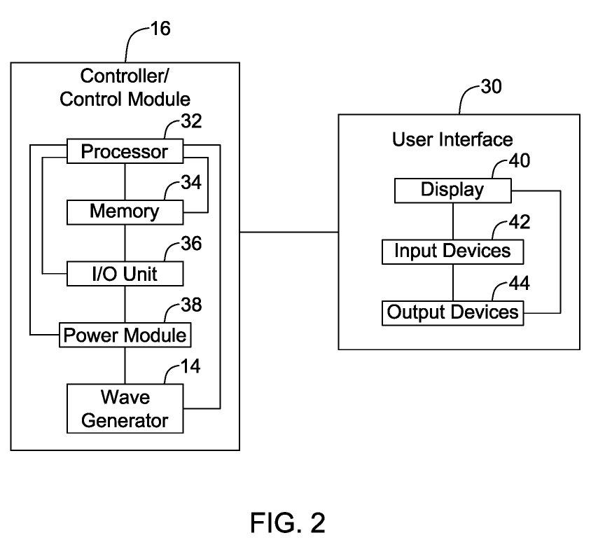

FIG. 2 depicts a schematic diagram of an illustrative configuration of the controller/control module 16 (e.g., a computing device) and a user interface 30 of the system 10. The controller 16 may be and/or may include any suitable computing device configured to process data of or for the system 10 and provide power to the one or more wave generators 14 via, in some examples, a power connector and/or electrical conductors. In some cases, one or more components of the system 10 may be incorporated into the controller 16 and/or the user interface 30. Further, one or more components of the system 10 may incorporate one or more computing devices similar to or having components similar to the controller 16 and/or the user interface 30.

The controller 16 may be configured to facilitate operation of the system 10. The controller 16, in some cases, may be configured to monitor and/or control an amount of power and/or a frequency of power applied to the one or more wave generators 14 according to a preconfigured control program, according to user interactions with the user interface 30, and/or in response to values from one or more sensors and/or values of one or more monitored metrics or parameters reaching or going beyond a threshold value. In some examples, the controller 16 may be configured to provide electrical current pulses (e.g., direct current (DC)), high voltage pulses, optical pulses, and/or other suitable pulses of power to the wave generators 14, but other suitable configurations are contemplated. The controller 16 may be in communication with a wall power source, a battery power source, a renewable energy power source, and/or other suitable source of power.

The controller 16 may communicate with a remote server or other suitable computing device. When the controller 16, or at least a part of the controller 16, is a component separate from a structure of the medical device 12, the controller 16 may communicate with electronic components of the system 10 over one or more wired or wireless connections or networks (e.g., LANs and/or WANs).

The controller 16 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs) or System On Chips (SOCs), software, hardware, firmware, or any combination of these and/or other components. Although the controller 16 may be referred to herein in the singular, the controller 16 may be implemented in multiple instances, distributed across multiple computing devices, instantiated within multiple virtual machines, and/or the like.

The illustrative controller 16 may include, among other suitable components, one or more processors 32, memory 34, one or more power modules 36, one or more input/output (I/O) units 38, and/or one or more wave generators 14. Although the wave generator 14 is depicted in FIG. 1 as being separate from the controller 16, the wave generator 14 may be part of the controller (e.g., as depicted in FIG. 2) or the controller 16 may be part of the wave generator 14. Example other suitable components of the controller 16 that are not specifically depicted in FIG. 2 may include, but are not limited to, communication components, a touch screen, selectable buttons, a housing, and/or other suitable components of a controller. As discussed above, one or more components of the controller 16 may be separate from the components of the system 10 and/or incorporated into the components of the system 10.

The processor 32 of the controller 16 may include a single processor or more than one processor working individually or with one another. The processor 32 may be configured to receive and execute instructions, including instructions that may be loaded into the memory 34 and/or other suitable memory. Example components of the processor 32 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 34 of the controller 16 may include a single memory component or more than one memory component each working individually or with one another. Example types of memory 34 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 34 may be or may include a transitory or non-transitory computer readable medium. The memory 34 may include instructions stored in a transitory and/or non-transitory state on a computer readable medium that may be executable by the processor 32 to cause the processor 32 to perform one or more of the methods and/or techniques described herein.

The power module 38 may include any suitable component configured to facilitate providing power to the one or more wave generators 14. Example suitable components of the power module 38 may include, but are not limited to, a DC-DC converter, a DC-AC converter, an optical converter (e.g., a component configured to convert electrical power to into optical power (e.g., a light or laser beam)), a high voltage capacitor, a transistor switch, a power measurement unit (e.g., voltage measurement unit, current measurement unit, optical power measurement unit, and/or other suitable measurement unit), a device identification unit (e.g., a unit that identifies whether the medical device is compatible with the controller 16), and/or other suitable components.

The I/O units 36 of the controller 16 may include a single I/O component or more than one I/O component each working individually or with one another. Example I/O units 36 may be or may include any suitable types of mechanical communication hardware, electrical communication hardware, optical communication hardware, and/or software including, but not limited to, power input ports to receive power from a power source, power output ports to provide power to the medical device 12, device ports for coupling with the medical device 12, communication ports configured to communicate with electronic components of the system 10 and/or with other suitable computing devices or systems. Example types of I/O units 36 may include, but are not limited to, wired power components, wired optical components, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless power components, wireless optical components, wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 36.

The user interface 30 may be configured to communicate with the controller 16 via one or more wired or wireless connections. The user interface 36 may include one or more display devices 40, one or more input devices 42, one or more output devices 44, and/or one or more other suitable features.

The display device 40 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 42 may be and/or may include any suitable components and/or features for receiving user input via the user interface. Example input device(s) 42 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 44 may be and/or may include any suitable components and/or features for providing information and/or data to users and/or other computing components. Example output device(s) 44 include, but are not limited to, displays, speakers, vibration systems, tactile feedback systems, optical outputs, cables, lights, and/or other suitable output devices.

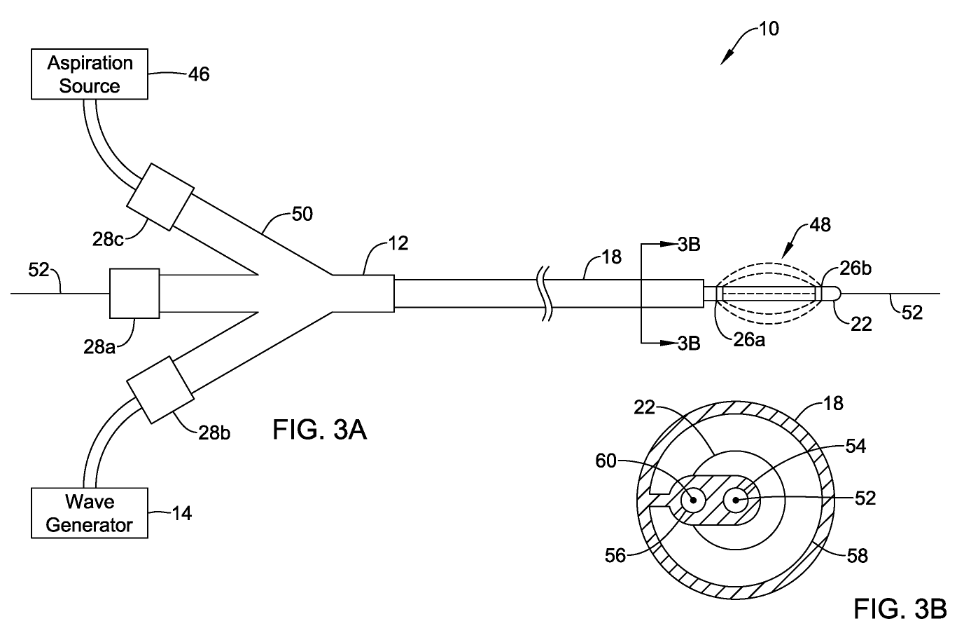

FIGS. 3A-8B depict schematic diagrams of illustrative configurations of the system 10. Components from one of the illustrative configurations of the system 10 may be utilized in other configurations of the system 10, unless expressly indicated otherwise. Although FIGS. 3A-8B do not depict the controller 16, the controller 16 may be in communication with and/or incorporated in the wave generator 14 and/or other components of the system 10.

FIGS. 3A and 3B schematically depict an illustrative configuration of the system 10 that may be configured to include and/or couple with an aspiration source 46. FIG. 3A depicts a schematic side view of the illustrative configuration of the system 10 and FIG. 3B depicts a schematic cross-section view taken along line 3B-3B in FIG. 3A. When the medical device 12 is coupled with the aspiration source 46, the aspiration source 46 may be configured to apply a negative pressure at a distal end of the first elongate shaft 18 to remove (e.g., suction or aspirate) fibrin from a thrombus after or during lysing of the RBCs of the thrombus via PFA applied by the electrodes 26 to cause and/or induce IRE in the RBCs. Although not depicted in FIG. 3A, the aspiration source 46 may be in communication with the controller 16 to facilitate control of the aspiration source 46, but other suitable configurations are contemplated.

The aspiration source 46 may be any suitable aspiration source 46 configured to couple with an aspiration lumen (e.g., a first lumen) of the medical device 12. Example suitable aspiration sources 46 include, but are not limited to, a mechanical fluid pump, an automated fluid pump, a syringe, a syringe actuation device, a power injector, a vacuum, a house vacuum, a stored vacuum, and/or other suitable aspiration sources.

As depicted in FIG. 3A, the second elongate shaft 22 may include a first electrode 26a and a second electrode 26b arranged in a bipolar configuration to create an electric field 48 therebetween. One of the first electrode 26a and the second electrode 26b may be a cathode and the other electrode may be an anode. In one example, the first electrode 26a may be an anode and the second electrode 26b may be a cathode, but other suitable configurations are contemplated. Although FIG. 3A depicts the second elongate shaft 22 with a single pair of electrodes 26 (e.g., a pair formed by the first electrode 26a and the second electrode 26b), the second elongate shaft 22 and/or the first elongate shaft 18 may include additional or alternative electrode pairs and/or electrodes 26 that may be configured in bipolar and/or monopolar arrangements or configurations.

The first electrode 26a and the second electrode 26b may have any suitable configuration configured to create a bipolar electric field 48 therebetween. In some examples and as depicted in FIG. 3A, the first electrode 26a and the second electrode 26b may be ring electrodes longitudinally spaced from one another to create the electric field 48 circumferentially around the second elongate shaft 22. Other suitable configurations of the first electrode 26a and the second electrode 26b are contemplated.

The electric field 48 may be configured to extend entirely and/or partially circumferentially around the second elongate shaft 22. In one example, the electrical field 48 may be configured to extend circumferentially around the elongate shaft a distance in range of 5% to 100% circumferentially around the elongate shaft 22, but other suitable ranges are contemplated. The electric field 48 may be configured to extend longitudinally along the elongate shaft 22 any suitable length. In one example, the electrical field 48 may be configured to extend longitudinally along the elongate shaft 22 a length in a range of 5 millimeters (mm) to 100 mm, but other suitable ranges are contemplated. In some examples, a circumferential and/or longitudinal extent of the electric field 48 may be adjustable by manipulating a sleeve extending over the elongate shaft 22 and/or may be adjustable in one or more other suitable manners.

In the configuration of the system 10 depicted in FIG. 3A, the second elongate shaft 22 may be fixed relative to the first elongate shaft 18. For example, the second elongate shaft 22 may be rotationally and/or longitudinally fixed relative to the first elongate shaft 18. Other configurations are contemplated in which the second elongate shaft 22 may be adjustable relative to the first elongate shaft 18.

The distal end or portion of the second elongate shaft 22 extending distal of the first elongate shaft 18 may have an outer diameter that is fixed so as not to be radially adjustable. In some examples and as depicted in FIG. 3A, the first electrode 26a and the second electrode 26b may be located along the fixed outer diameter of the second elongate shaft 22, but other suitable configurations are contemplated.

The medical device 12 may be coupled with one or more other components of the system 10. As depicted in the configuration of the system 10 of FIG. 3A, the medical device 12 may be coupled with the wave generator 14 and an aspiration source 46. In some examples, the medical device 12 may include a hub 50 for coupling with the wave generator 14 and the aspiration source 46 via any suitable coupling technique.

The hub 50 may include the one or more ports 28 of the medical device 12. In some examples, the hub 50 may include a first port 28a in communication with a guidewire lumen that is configured to receive a guidewire 52, a second port 28b in communication with a wire/lead lumen and configured to couple with the wave generator 14, and a third port 28c in communication with an aspiration lumen and configured to couple with the aspiration source 46. The guide wire lumen extending from the first port 28a, the wire/lead lumen extending from the second port 28b, and the aspiration lumen extending from the third port 28c may extend through and/or may be defined by the hub 50, the first elongate shaft 18, and/or the second elongate shaft 22.

Although the medical device 12 is depicted as a medical device 12 that is configured to be delivered to a target area using an over over-the-wire technique via a guidewire port (e.g., the first port 28a) at the hub 50, the medical device 12 may be configured to be delivered to a target area in one or more other suitable manners. For example, the medical device 12 may be configured to be delivered to a target area through a guide tube and/or over a guidewire via a rapid-exchange technique using a side port in the first elongate shaft 18 that may be distal of the hub 50.

As depicted in FIG. 3A, the hub 50 may be a single component coupled with the first elongate shaft 18 and/or other suitable components of the medical device 12. Alternatively, the hub 50 may include one or more components configured to couple together or be used together, where the one or more components of the hub 50 may each include one or more ports 28.

The cross-section of the medical device 12 schematically depicted in FIG. 3B shows one or more lumens of the medical device 12 and other components of the system 10. For example, FIG. 3B depicts an end view of the second elongate shaft 22 with an aspiration lumen 58 at least partially defined by the first elongate shaft 18 and having a larger inner diameter than an outer diameter of the second elongate shaft 22. Further, the first elongate shaft 18 may, at least in part, define a portion of a guidewire lumen 54 and a wire/lead lumen 56. The guidewire 52 may extend through the guidewire lumen 54 and one or more wires/leads 60 may extend through the wire/lead lumen 56 extending between the wave generator 14 and the electrodes 26 (e.g., the first electrode 26a and the second electrode 26b).

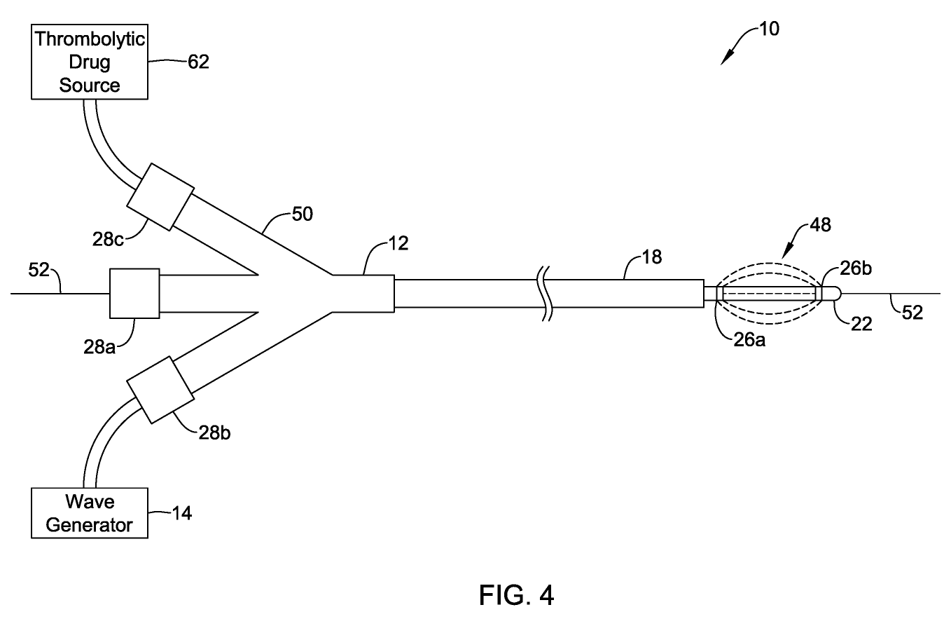

FIG. 4 schematically depicts an illustrative configuration of the system 10 that may be configured to include and/or couple with a thrombolytic drug source 62. As depicted in FIG. 4, the medical device 12 of the system 10 may be configured the same as or similar to the illustrative configuration of the medical device 12 depicted in FIGS. 3A and 3B, where the third port 28c may be a thrombolytic drug port in communication with a thrombolytic drug lumen (e.g., a first lumen) of the medical device 12 configured to receive a thrombolytic drug from the thrombolytic drug source 62. When the medical device 12 is coupled with the thrombolytic drug source 62, the thrombolytic drug source 62 may be configured to apply a thrombolytic drug to and/or out of a distal end of the first elongate shaft 18 to lyse fibrin from a thrombus before, after, and/or during lysing of the RBCs of the thrombus via PFA applied by the electrodes 26 to cause and/or induce IRE in the RBCs. Although not depicted in FIG. 4, the thrombolytic drug source 62 may be in communication with the controller 16 to facilitate control of the thrombolytic drug source 62, but other suitable configurations are contemplated.

The thrombolytic drug source 62 may be any suitable thrombolytic drug source 62 configured to couple with a drug delivery lumen of the medical device 12. Example suitable thrombolytic drug sources 62 include, but are not limited to, a mechanical drug pump, an automated drug pump, a syringe, a syringe actuation device, and/or other suitable thrombolytic drug sources.

The thrombolytic drug may be any suitable thrombolytic drug configured to lyse fibrin from a thrombus before, after, and/or during lysing of the RBCs of the thrombus. Example suitable thrombolytic drugs include, but are not limited to, streptokinase, alteplase, reteplase, tecteplase, urokinase, prourokinase, anistreplase, and/or other suitable thrombolytic drugs.

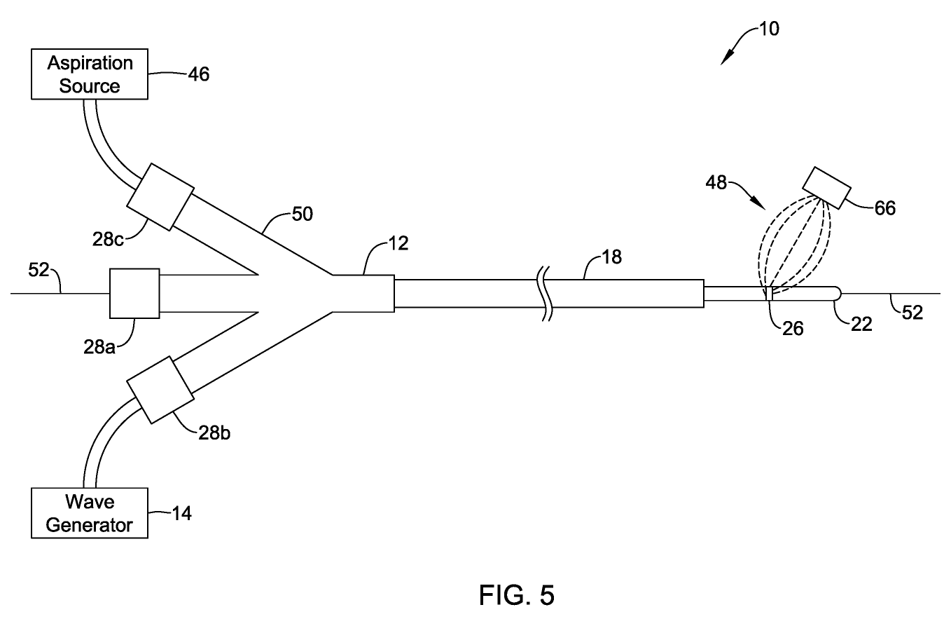

FIG. 5 schematically depicts an illustrative configuration of the system 10 that may include an electrode 26 having a monopolar configuration. As depicted in FIG. 5, the system 10 may be configured the same as or similar to the illustrative configuration of the system 10 depicted in FIGS. 3A and 3B, where the electrode configuration at the distal end or portion of the second elongate shaft 22 may include a single electrode 26 configured to create a monopolar electric field.

The electrode 26 at the distal end or portion of the second elongate shaft 22 may have any suitable configuration configured to create a monopolar electric field 48 with a grounding pad 66. In some examples, the electrode 26 and the second elongate shaft 22 may be located in a vessel of a patient and the grounding pad 66 may be located exterior of the patient. Creating the monopolar electric field between the electrode 26 and the grounding pad 66 may allow a user to manipulate a location of the electrode 26 by adjusting relative positions of the electrode 26 and the grounding pad 66. In some examples, the electrode 26 configured to create the monopolar electric field 48 may be a ring electrode located at the distal end or portion of the second elongate shaft 22. Other suitable configurations of the electrode 26 are contemplated.

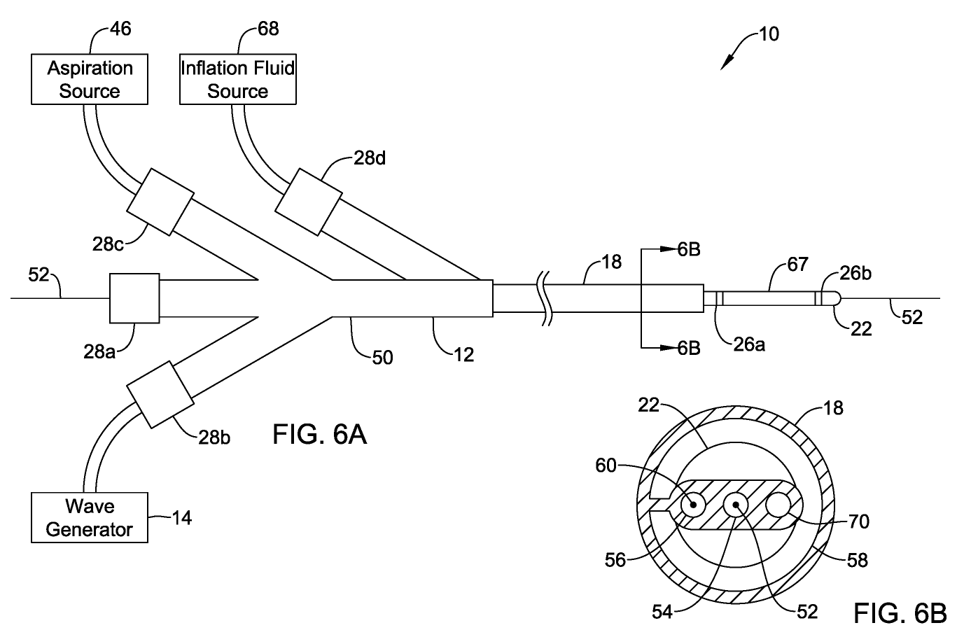

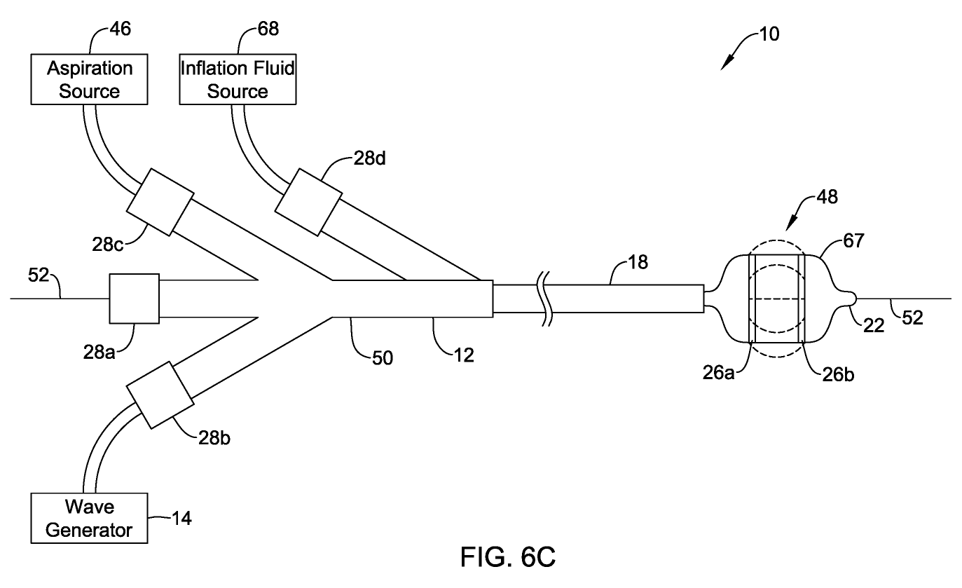

FIGS. 6A-6C schematically depict an illustrative configuration of the system 10 that may be configured to include and/or couple with the aspiration source 46 and an inflation fluid source 68, where the inflation fluid source 68 may be configured to provide fluid flow for expanding and/or contracting an expandable member 67. As depicted in FIG. 6A, the medical device 12 of the system 10 may be configured the same as or similar to the illustrative configuration of the medical device 12 depicted in FIGS. 3A and 3B, where the hub 50 may include a fourth port 28d and the second elongate shaft 22 may include the expandable member 67. In some examples, the fourth port 28d may be an inflation fluid port in communication with an inflation lumen and configured to couple with the inflation fluid source 68.

When the medical device 12 is coupled with the inflation fluid source 68, the inflation fluid source 68 may be configured to control an amount of fluid provided to the expandable member 67 via the inflation lumen to inflate and/or deflate the expandable member 67. For example, the inflation fluid source 68 coupled with the fourth port 28d may provide fluid to the expandable member 67 to expand the expandable member 67 prior to or during the application of PFA energy to a thrombus to alter engagement of the electric field with the RBCs of the thrombus and to remove fluid from the expandable member 67 during and/or after applying the PFA energy to the thrombus, such that the expandable member 67 may be deflated prior to removing the first elongate shaft 18 and/or the second elongate shaft 22 from the vessel of the subject.

The inflation fluid source 68 may be any suitable inflation fluid source 68 configured to couple with an inflation lumen of the medical device 12. Example suitable inflation fluid sources 68 include, but are not limited to, a mechanical fluid fluid pump, an automated fluid pump, a syringe, a syringe actuation device, and/or other suitable inflation fluid sources. Although not depicted in FIG. 6A, the inflation fluid source 68 may be in communication with the controller 16 to facilitate control of the inflation fluid source 68, but other suitable configurations are contemplated.

The cross-section of the medical device 12 schematically depicted in FIG. 6B shows lumens of the medical device 12 and other components of the system 10. For example, the medical device 12 depicted in FIG. 6B depicts an end view of the second elongate shaft 22 with an aspiration lumen 58 at least partially defined by the first elongate shaft 18 and having a larger inner diameter than an outer diameter of the second elongate shaft 22. Further, the first elongate shaft 18 may define at least a portion of the guidewire lumen 54 with the guidewire 52 extending therethrough, a portion of the wire/lead lumen 56 with the one or more wires/leads 60 extending therethrough, and a portion of an inflation lumen 70 configured to transfer inflation fluid between the inflation fluid source 68 and the expandable member 67. Although the inflation lumen 70 is depicted as being defined by the first elongate shaft 18 in FIG. 6B, the inflation lumen 70 may be defined, at least in part, by the hub 50, the second elongate shaft 22, and/or other components of the system 10. In one example, the second elongate shaft 22 may extend from a location distal of the first elongate shaft 18 to the hub 50 and define the inflation lumen 70 therebetween such that the first elongate shaft 18 does not define the inflation lumen 70. Other suitable configurations of the lumens of the medical device 12 are contemplated.

FIG. 6C is a schematic view of the system 10, with the expandable member 67 in an expanded configuration and a bipolar electric field 48 extending between the first electrode 26a and the second electrode 26b. When the electrodes 26 are located on the expandable member 67, the electrodes 26 may be configured to expand with the expandable member 67 or may be configured to maintain a shape and/or size as the expandable member 67 expands. In one example and as depicted in FIG. 6C, the first electrode 26a and the second electrode 26b may be flexible ring electrodes configured to expand (and contract) with the expandable member 67.

Although only a single expandable member 67 having a single expandable lobe is depicted in FIGS. 6A-6C, a plurality of expandable members 67 and/or an expandable member 67 having a plurality of lobes may be utilized. Utilizing a plurality of expandable members 67 and/or a plurality of lobes of the expandable member 67 may facilitate blocking a blood flow through the vessel during a procedure, contouring the expandable member(s) 67 to match a shape of a vessel or thrombus, contouring the expandable member(s) 67 to direct the electric field(s) 48 from electrodes 26 thereon toward a thrombus, and/or may be provided for other suitable purposes.

In addition to or as an alternative to the inflation fluid source 68, one or more wires or control components coupled with the expandable member 67 may extend from the hub 50. The one or more wires or control components may be adjusted or manipulated to adjust a position or orientation of the expandable member. In one example, a wire extending through a lumen in communication with the fourth port 28d or other suitable port 28 may be coupled with a distal end of the expandable member 67 and adjusting the wire in a proximal direction may radially expand the expandable member 67 to a second orientation and adjusting the wire in a distal direction may radially contract the expandable member 67 to a first orientation. Other suitable configurations of the wire coupled with the expandable member 67 are contemplated.

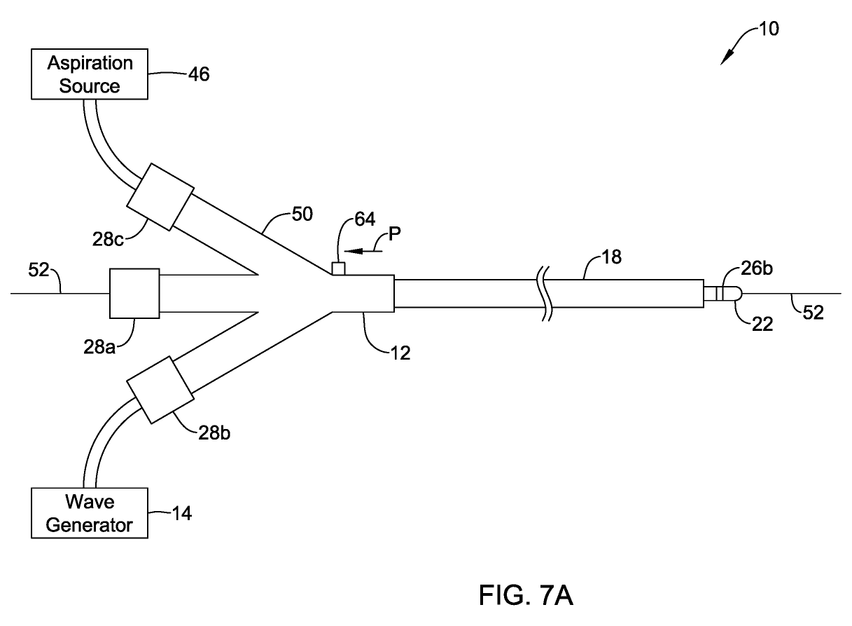

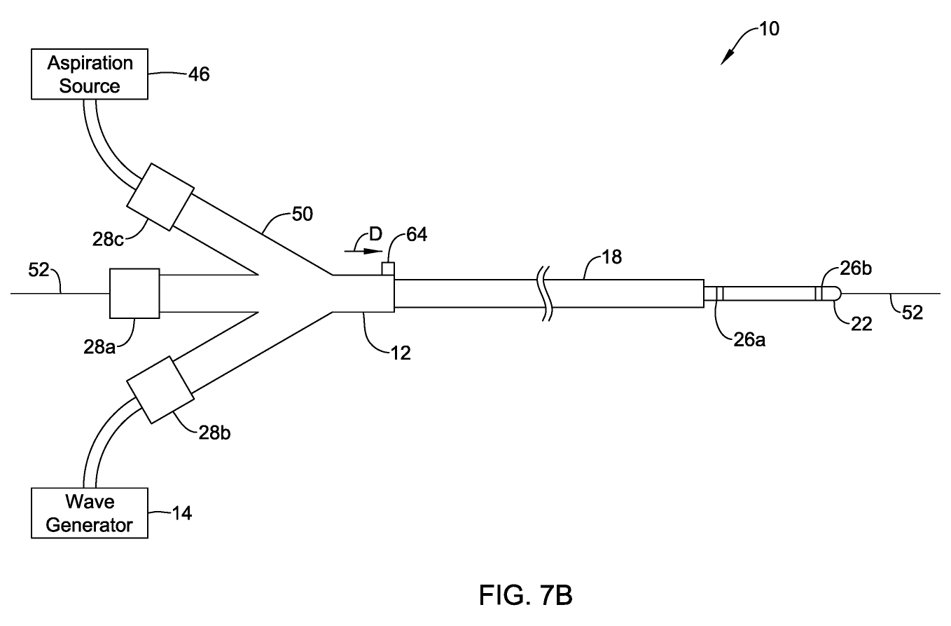

FIGS. 7A and 7B schematically depict an illustrative configuration of the system 10, where the second elongate shaft 22 may be adjustable (e.g., longitudinally and/or rotationally) relative to the first elongate shaft 18 and the second elongate shaft may include a bipolar configuration of electrodes 26. As depicted in FIGS. 7A and 7B, the second elongate shaft 22 may be an inner shaft and the first elongate shaft 18 may be an outer shaft. The hub 50 may include an actuator 64 coupled with the second elongate shaft 22.

The actuator 64 may be configured to adjust (e.g., rotationally, longitudinally, and/or circumferentially) relative to the hub 50 and as the actuator 64 adjusts relative to the hub 50, the second elongate shaft 22 may adjust with the actuator 64 relative to the hub 50 and/or the first elongate shaft 18. The actuator 64 may be any suitable type of actuator including, but not limited to, a wheel, a slider a knob, a tab, a pin, a roller, and/or other suitable actuator configured to adjust relative to the hub 50.

The actuator 64 may be coupled with the second elongate shaft 22 in any suitable manner. For example, the actuator 64 may be coupled with the second elongate shaft 22 via a connecting shaft, a tube, a hypotube, a gear, a rack and a pinion, a wire, a threaded connection, and/or via one or more other suitable coupling features.

As depicted in FIG. 7A, the actuator 64 may be a knob in a proximal position and the second elongate shaft 22 may be advanced in a proximal direction P relative to the hub and/or the first elongate shaft 22. When the actuator 64 is advanced in the proximal direction P, at least a portion of the distal end or distal portion of the second elongate shaft 22 may be positioned within a lumen of the first elongate shaft 18. As depicted in FIG. 7A, the first electrode 26a (not shown) may be located within the first elongate shaft 18 and the second electrode 26b may be located distal of the first elongate shaft.

As depicted in FIG. 7B, the actuator 64 may be in a distal position and the second elongate shaft 22 may be advanced in a distal direction D relative to the hub 50 and/or the first elongate shaft 18. When the actuator 64 is advanced in the distal direction D, the first electrode 26a and the second electrode 26b may be located distal of a distal end of the first elongate shaft 18. As such and in some examples, the actuator 64 may be adjusted to adjust a position of the electrodes on the second elongate shaft 22 relative to the first elongate shaft 18 to control a strength, depth, and/or direction of the electric field from the electrodes 26. In some examples, adjusting a longitudinal and/or rotational position of the second elongate shaft 22 may facilitate inserting the second elongate shaft 22 in the thrombus, creating a desired apposition between the second elongate shaft 22 (e.g., and the electrodes 26 thereon) and the thrombus in a vessel of the subject, and/or directing an electric field toward a target area.

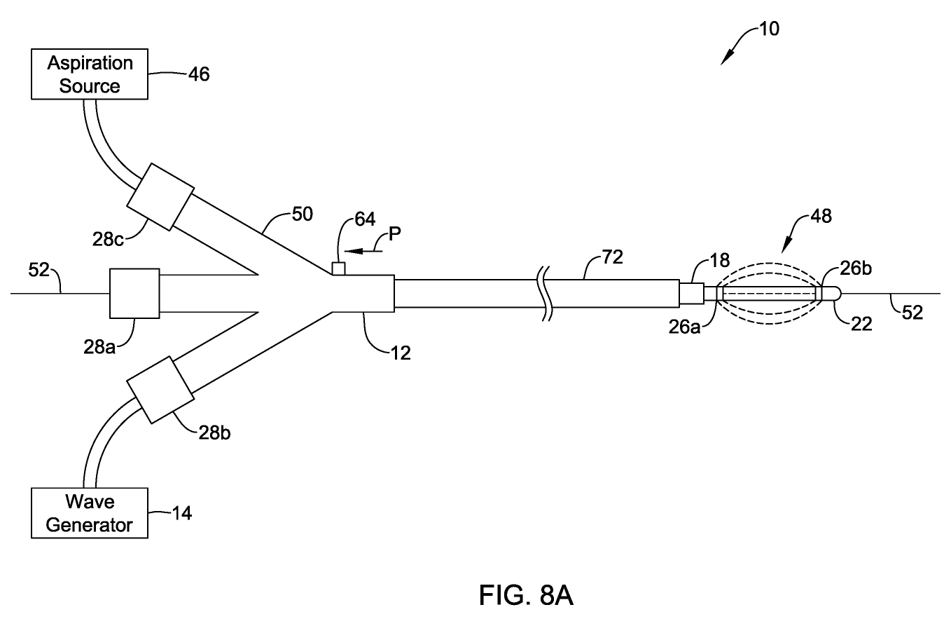

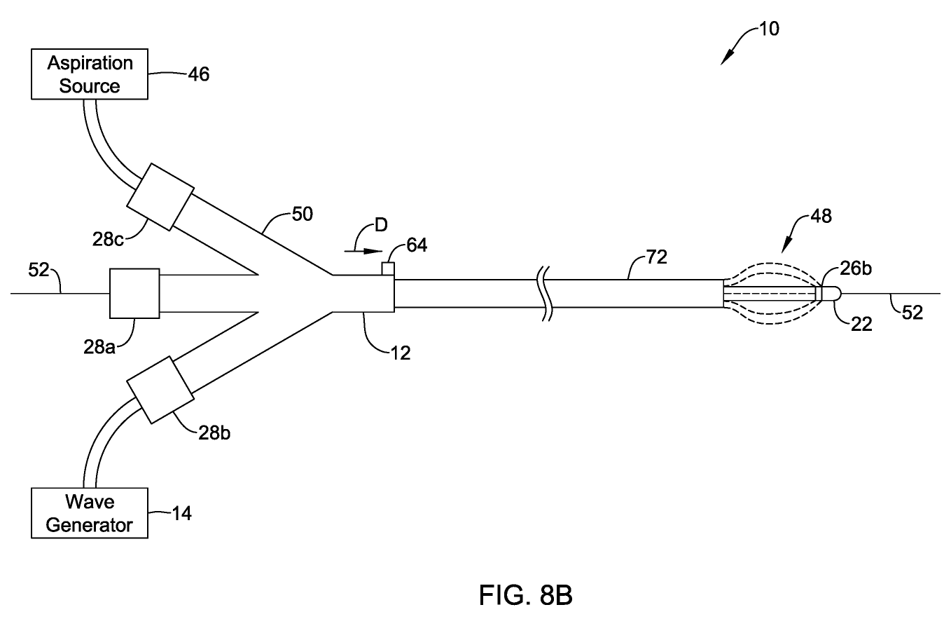

FIGS. 8A and 8B schematically depict an illustrative configuration of the system 10, where the system comprises a sleeve 72 configured to adjust (e.g., longitudinally and/or rotationally) relative to the first elongate shaft 18 and the second elongate shaft 22. The system 10 depicted in FIGS. 8A and 8B may be the same as or similar to the configuration of the system 10 depicted in FIGS. 7A and 7B, except the actuator 64 may be coupled with the sleeve 72 instead of with the second elongate shaft 22 and the sleeve 72 may adjust with or in response to adjustment of the actuator 64.

The actuator 64 may be configured to adjust (e.g., rotationally, longitudinally, and/or circumferentially) relative to the hub 50 and as the actuator 64 adjusts relative to the hub 50, the sleeve 72 may adjust with the actuator 64 relative to the hub 50 and/or the first elongate shaft 18. The actuator 64 may be coupled with the sleeve 72 in any suitable manner. For example, the actuator 64 may be coupled with the sleeve 72 via a connecting shaft, a tube, a hypotube, a gear, a rack and a pinion, a wire, a threaded connection, and/or via one or more other suitable coupling features.

The sleeve 72 may be formed from any suitable material. In some examples, the sleeve 72 may be formed from a material configured to create or permit a dampening effect on a generated electric field. Example suitable materials for use in forming the sleeve include, but are not limited to, electrically insulative materials, electrical field dampening materials, polytetrafluoroethylene (PTFE), fluorinated ethylene propylene (FEP), polyamide with polyether blocks, and/or other suitable materials.

As depicted in FIG. 8A, the actuator 64 may be a knob in a proximal position and the sleeve 72 may be advanced in a proximal direction P relative to the hub 50, the first elongate shaft 18, and/or the second elongate shaft 22. When the actuator 64 is advanced in the proximal direction P, the distal end or distal portion of the second elongate shaft 22 at which the electrodes 26 are located may be exposed. For example and as depicted in FIG. 8A, the second elongate shaft 22 may include the first electrode 26a and the second electrode 26b with an electric field 48 extending therebetween.

As depicted in FIG. 8B, the actuator 64 may be in a distal position and the sleeve 72 may be advanced in the distal direction D relative to the hub 50, the first elongate shaft 18, and/or the second elongate shaft 22. When the actuator 64 is advanced in the distal direction D, the sleeve 72 may extend over a portion of the second elongate shaft 22 extending distal of the first elongate shaft 18. In some examples, the sleeve 72 may be an electrical insulator and may extend over the second elongate shaft 22 and cover at least a portion of the first electrode 26a to shape, orient, and/or control the electric field 48 extending between the first electrode 26a (not shown in FIG. 8B) and the second electrode 26b. In some examples, the sleeve 72 may block electrons from moving between the first electrode 26a and 26b and/or may extend over the first electrode 26a and the second electrode 26 to dampen the electric field 48 or block the electric field 48 from reaching target cells of the subject.

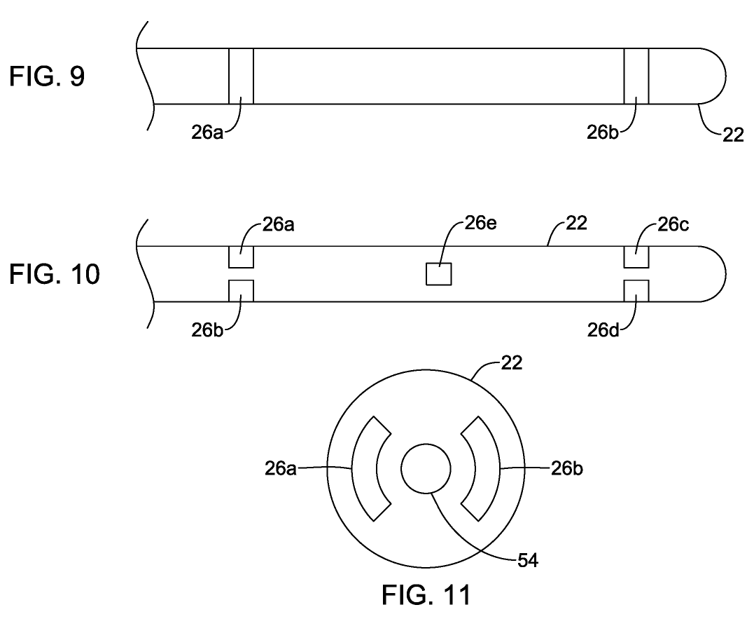

FIGS. 9-11 depict illustrative configurations of electrodes 26 on the second elongate shaft 22. Although certain configurations of the electrodes 26 are depicted in FIGS. 9-11, other suitable configurations of the electrodes 26 are contemplated.

FIG. 9 depicts a schematic side view of an illustrative configuration of the first electrode 26a and the second electrode 26b configured as ring electrodes on the second elongate shaft 22. As discussed herein, the first electrode 26a and the second electrode 26b may be configured to form a bipolar electric field 48 configured to apply PFA voltage pulses to RBCs of a thrombus.

FIG. 10 depicts a schematic side view of an illustrative configuration of the first electrode 26a, the second electrode 26b, a third electrode 26c, a fourth electrode 26d, and a fifth electrode 26e, all on the second elongate shaft 22. In some examples, the first electrode 26a and the second electrode 26b may be discrete electrodes located at a same first axial location, but different circumferential locations, on the second elongate shaft 22 and may form a bipolar electric field therebetween. In some examples, the third electrode 26c and the fourth electrode 26d may be discrete electrodes located at a same second axial location, but different circumferential location, on the second elongate shaft 22 and may form a bipolar electric field therebetween. The second axial location may be distal of the first axial location, but other suitable configurations are contemplated. The fifth electrode 26e may be located between the first axial location and the second location. The fifth electrode 26e may be a cathode and configured to form a bipolar connection with one or both of an anode electrode at the first axial location and the second axial location. Alternatively or additionally, the fifth electrode 26e may be in a monopolar arrangement and configured to create a monopolar electric field with the grounding pad 66 external of the subject and/or other suitable grounding pad.

FIG. 11 depicts a schematic end view of the first electrode 26a and the second electrode 26b configured as arc-shaped electrodes on a distal end of the second elongate shaft 22 defining, at least in part, the guidewire lumen 54. In some examples, the first electrode 26a and the second electrode 26b may be sized, shaped, and/or positioned to create a forward-facing electric field (e.g., an electric field that extends in a distal direction from the second elongate shaft 22). For example, the first electrode 26a and the second electrode 26b depicted in FIG. 11 may be configured to create a forward-facing electric field. In some examples, utilizing a forward-facing electric field may facilitate weakening a portion of a thrombus (e.g., via lysing RBCs using IRE) proximate the distal end of the second elongate shaft 22 to facilitate inserting the medical device 12 into the thrombus for further application of one or more PFA electric fields to the RBCs of the thrombus.

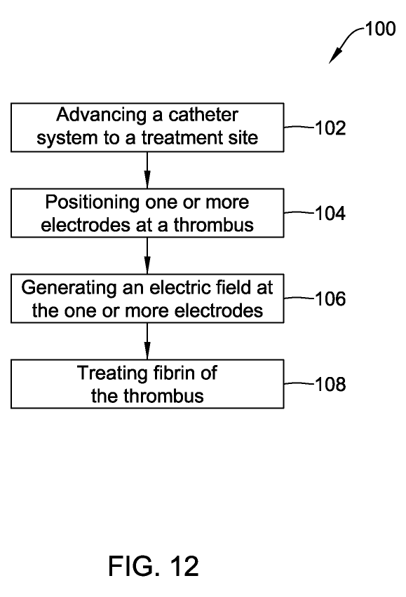

FIG. 12 depicts a schematic diagram of an illustrative technique or method 100 for treating a thrombus (e.g., debulking a thrombus). In some examples, the technique or method 100 may utilize pulse field ablation (PFA) voltage pulses to cause irreversible electroporation (IRE) in red blood cells (RBCs) of the thrombus to lyse the RBC. After lysing the RBCs, any remaining fibrin of the thrombus may be removed from the vessel and/or lysed.

The method 100 may include advancing 102 a catheter system to a treatment site within a vessel of a subject (e.g., a patient). In some examples, the treatment site (e.g., a target site) may be located in a peripheral vein of the subject, but the treatment site may be located in one or more other vessels of the subject. The treatment site may include or contain a thrombus and/or other blockage in the vessel.

The catheter system may have any suitable configuration configured to facilitate applying an electric field to the thrombus and/or other blockage in the vessel at the treatment site. The catheter system may be configured similar to the systems discussed herein and/or may be configured in other suitable manners. In some examples, the catheter system may include an elongate shaft with one or more electrodes disposed along a distal portion of the elongate shaft and the catheter system may be manipulated to position 104 the one or more electrodes in and/or at the thrombus. In some examples, the catheter system may include one or more expandable members at a distal end of the elongate shaft along which the one or more electrodes may be located (e.g., the one or more electrodes may position a longitudinal location of the expandable member) and positioning 104 the one or more electrodes in and/or at the thrombus may include radially expanding the expandable member at and/or within the thrombus. The catheter system may have one or more other suitable configurations.

Once the one or more electrodes are positioned at the thrombus and/or as the one or more electrodes are being positioned at the thrombus, an electric field may be generated 106 at the one or more electrodes. In some examples, the generated electric field may be configured to apply or deliver PFA energy (e.g., PFA voltage pulses) to the thrombus and/or other suitable voltage pulses to the thrombus. In one example, the generated electric field may be configured to apply or deliver PFA voltage pulses to the thrombus that are configured to induce IRE in RBCs of the thrombus to lyse the RBCs. For example, an electric field configured to deliver PFA voltages to the thrombus that are greater than 1100-1200 volts (V) per centimeter (cm) (V/cm) for 100-300 microseconds (µs) may be configured to induce IRE in RBCs. Other suitable generated voltage pulses are contemplated.

In some examples, sensing capabilities may be utilized to assist in applying an electric field to the thrombus. For example, values obtained from sensing pressure, impedance, temperature and/or other parameters may be used in controlling the generated electric to induce IRE in RBCs. In some examples, values of sensed parameters may used in a closed-loop control for generating the electric field.

After generating 106 the electric field and/or lysing the RBCs of the thrombus, any remaining fibrin of the thrombus may be treated 108 to fully remove the thrombus from the vessel of the subject. In some examples, the remaining fibrin of the thrombus may be treated by aspirating the fibrin from the vessel using aspiration components of the catheter system and/or other suitable aspiration systems. In some examples, the remaining fibrin of the thrombus may be treated by applying a thrombolytic drug to fibrin and lysing the fibrin with the thrombolytic drug. Other suitable techniques for treating the fibrin of the thrombus are contemplated.

In some examples, a fluid may be delivered to the treatment site or thrombus prior to and/or while generating the electric field. The fluid may facilitate transferring voltage pulses of the electric field to the RBCs of the thrombus. In some examples, the fluid delivered to the treatment site or thrombus to facilitate transferring voltage pulses of the electric field to the RBCs of the thrombus may be electrically conductive fluids, but other suitable fluids are contemplated. The fluid, however, may be omitted.

When utilized, any suitable electrically conductive fluid may be delivered to the treatment site or thrombus. Example suitable electrically conductive fluids include, but are not limited to, saline, pure-water mixture (0.1%-5% of non-pure-water), polar or ionic fluids that can carry free ions, calcium chloride, and/or other suitable fluids

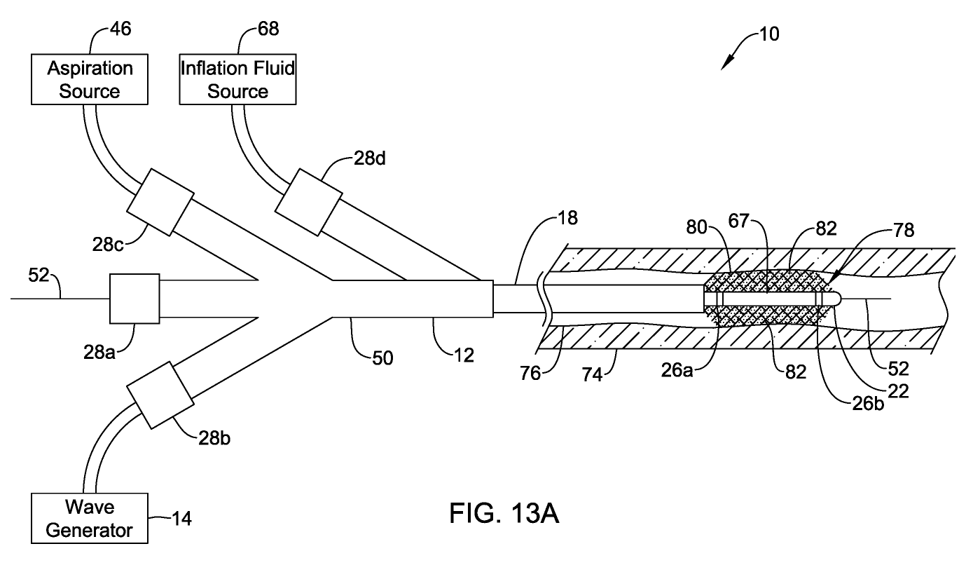

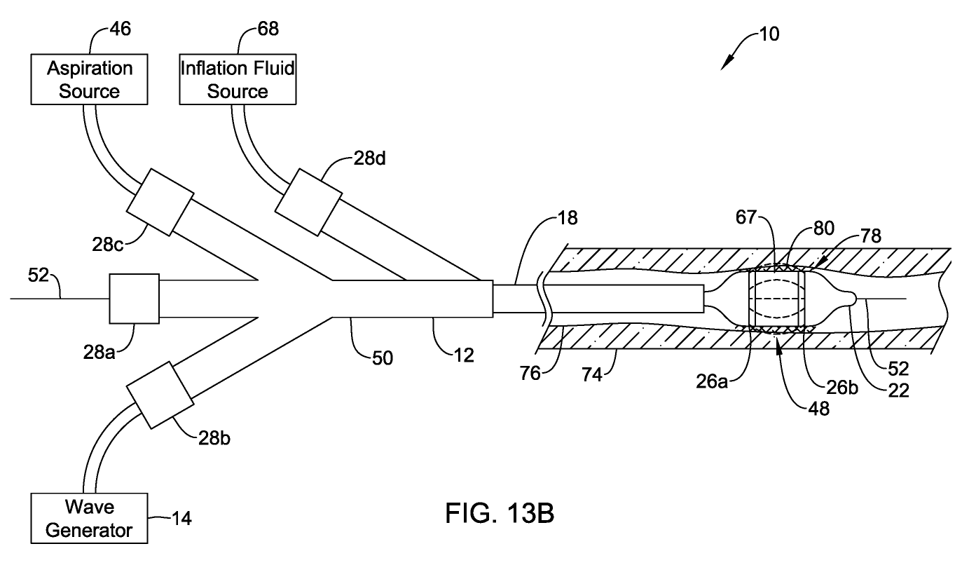

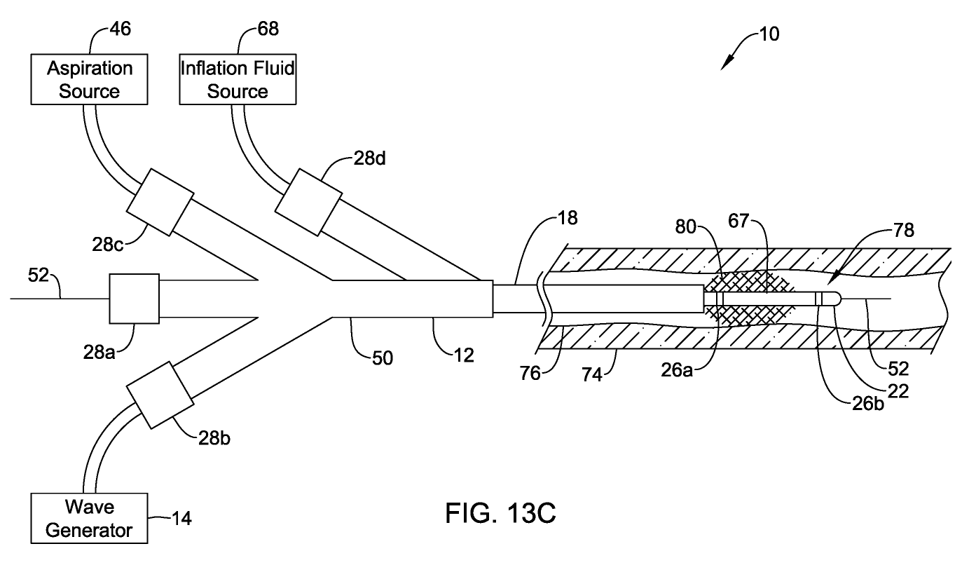

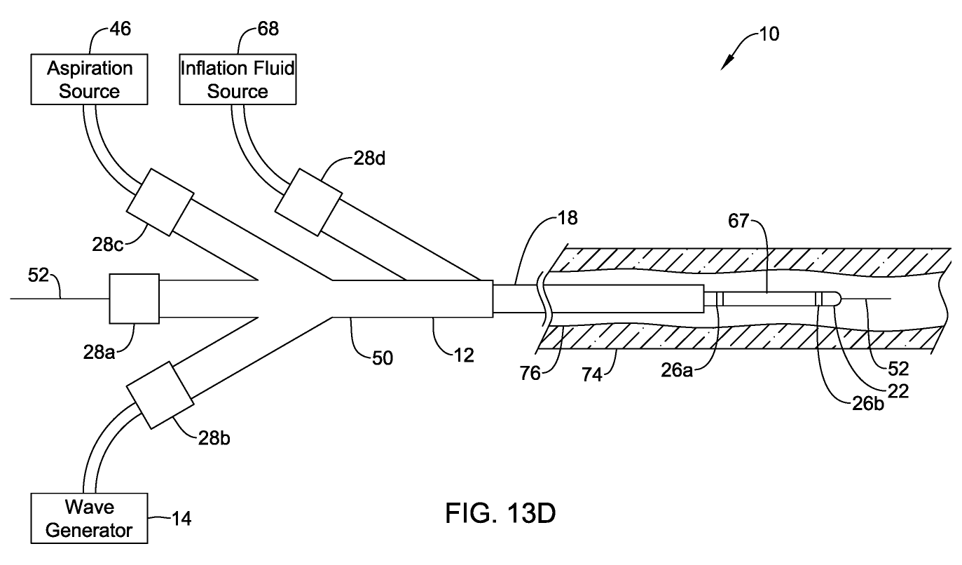

FIGS. 13A-13D schematically depict illustrative steps in a technique or method of treating a thrombus using the system 10. FIGS. 13A-13D depict the illustrative configuration of the system 10 of FIGS. 6A-6C having the medical device 12 with the expandable member 67 with the first electrode 26a and the second electrode 26b disposed on the expandable member 67. Similar steps to those depicted in FIGS. 13A-13D, however, may be performed with other configurations of the system 10.

FIG. 13A depicts the first elongate shaft 18 and the second elongate shaft 22 of the medical device 10 delivered over the guidewire 52 to a treatment site, where a thrombus 78 is located in a lumen 76 of a vessel 74. In some examples, the thrombus 78 may be formed of fibrin 80 and red blood cells (RBCs) 82. In some configurations, the portion of the second elongate shaft 22 configured to create an electric field to apply PFA energy to the thrombus 78 may be positioned within the thrombus 78. For example, a portion of the second elongate shaft 22 comprising the expandable member 67, the first electrode 26a, and the second electrode 26b may be inserted into the thrombus 78.

FIG. 13B depicts the second elongate shaft 22 within the thrombus 78 and the expandable member 67 expanded to move the first electrode 26a and the second electrode 26b further into the thrombus 78 than prior to expansion of the expandable member 67. When the first electrode 26a and the second electrode 26b are positioned within the thrombus 78, the wave generator 14 may be initiated (e.g., via the controller 16 and/or initiated in one or more other manners) to send voltage pulses (e.g., PFA voltage pulses) to the first electrode 26a and/or the second electrode 26b to create the electric field 48 therebetween. As discussed, the voltage pulses applied to the thrombus 78 by the electric field 48 may be configured to induce IRE in the RBCs and lyse the RBCs 82 from the thrombus 78. The lysing of the RBCs from the thrombus 78 may thin the thrombus 78 of material, which is represented in FIG. 13B by showing the thrombus 78 containing the fibrin 80 and not the RBCs 82. Although the thrombus 78 is depicted without any RBCs 82 remaining therein, it is contemplated some RBCs 82 may remain in the thrombus 78 after the application of the electric field 48.

FIG. 13C depicts the second elongate shaft 22 within the thrombus 78, the expandable member 67 contracted, and no electric field 48 between the first electrode 26a and the second electrode 26b. Further, the aspiration source 46 has been activated and is aspirating the fibrin 80 of the thrombus 78 from the lumen 76 of the vessel 74 to fully treat the thrombus 78. Lysing or removing the RBCs 82 from the thrombus 78 may weaken the thrombus 78 and allow the fibrin 80 to collapse for or during removal. In addition to or as an alternative to aspirating the fibrin 80 from the vessel 74, the fibrin may be lysed to fully treat the thrombus 78.

FIG. 13D depicts the first elongate shaft 18 and the second elongate shaft 22 in the lumen 76 of the vessel 74 with the thrombus 78 fully treated and removed from the vessel 74. Once the thrombus has been fully treated, the medical device 12 may be removed from the subject. The techniques discussed with respect to FIGS. 12-13D that utilize PFA energy to induce IRE in RBCs may be configured to mitigate pain to the subject and/or damage or trauma to the vessels of the subject by mitigating a magnitude of force required for removing a thrombus from a vessel and/or an amount of drug needed to lyse the thrombus.

The materials that can be used for the various components of pulse field ablation (PFA) system 10 and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system, comprising:
a medical device comprising:
an elongate shaft; and
one or more electrodes disposed at a distal portion of the elongate shaft;
a wave generator configured to be in electrical communication with the one or more electrodes; and
a controller configured to selectively cause the wave generator to deliver pulse field ablation energy to an electrode of the one or more electrodes, the pulse field ablation energy is configured to lyse red blood cells of a thrombus in a vessel of a subject.

2. The system of claim 1, wherein the elongate shaft is a second elongate shaft and the medical device comprises a first elongate shaft having a lumen configured to pass fluid to treat a thrombus in a vessel of a subject.

3. The system of claim 1, further comprising:
an aspiration source configured to be in fluid communication with a lumen of the medical device to remove fibrin of the thrombus from the vessel of the subject in response to actuation of the aspiration source.

4. The system of claim 1, further comprising:
a source of a thrombolytic drug configured to be in fluid communication with a lumen of the medical device to lyse fibrin of the thrombus in the vessel of the subject with the thrombolytic drug in response to actuation of the source of the thrombolytic drug.

5. The system of claim 1, wherein the one or more electrodes include at least two electrodes in a bipolar arrangement along the elongate shaft.

6. The system of claim 1, wherein the one or more electrodes include an electrode in a monopolar arrangement along the elongate shaft.

7. The system of claim 6, further comprising:
a grounding pad configured to be located external of the subject and create an electric field with the electrode in the monopolar arrangement along the elongate shaft.

8. The system of claim 1, wherein the elongate shaft comprises an expandable member at the distal portion and the one or more electrodes are disposed at a longitudinal location of the expandable member.

9. The system of claim 8, wherein the medical device further comprises a lumen configured to facilitate radial adjustment of the expandable member.

10. The system of claim 9, further comprising:
an inflation fluid source fluidly coupled with the lumen of the medical device,
wherein the expandable member is a balloon configured to receive inflation fluid from the inflation fluid source via the lumen of the elongate shaft.

11. The system of claim 1, further comprising:
an insulative sleeve configured to be adjustably disposed over a portion of the elongate shaft to control a location of an electric field from the one or more electrodes.

12. A medical device, comprising:
a first elongate shaft comprising a first lumen, the first elongate shaft is configured to pass through a vessel of a subject and the first lumen is configured to pass fluid to treat a thrombus in the vessel of the subject;
a second elongate shaft configured to extend in a distal direction from a distal end of the first elongate shaft; and
one or more electrodes disposed at a distal portion of the second elongate shaft,
wherein the one or more electrodes are configured to apply pulse field ablation (PFA) energy to the thrombus to lyse red blood cells of the thrombus.

13. The medical device of claim 12, wherein the distal portion of the second elongate shaft comprises an expandable member and the one or more electrodes are disposed along a longitudinal location of the expandable member.

14. The medical device of claim 12, wherein the one or more electrodes include a plurality of electrodes configured in a bipolar arrangement.

15. The medical device of claim 12, further comprising an insulative sleeve disposed on a portion of the second elongate shaft to control a degree of electrode contact and length of longitudinal treatment using the one or more electrodes.

16. A method for treating a thrombus, comprising:
advancing a catheter system to a treatment site in a vessel containing a thrombus, the catheter system comprising an elongate shaft with one or more electrodes disposed along a distal portion of the elongate shaft;
positioning the one or more electrodes at the thrombus;
generating an electric field at the one or more electrodes to deliver pulse field ablation (PFA) energy to red blood cells of the thrombus and cause irreversible electroporation of the red blood cells; and
treating fibrin of the thrombus after the PFA energy is delivered to the red blood cells of the thrombus.

17. The method of claim 16, further comprising:
delivering a conductive fluid to the treatment site prior to delivering the PFA energy to the red blood cells of the thrombus.

18. The method of claim 16, wherein:
the elongate shaft includes an expandable member and the one or more electrodes are positioned at a longitudinal location of the expandable member, and
positioning the one or more electrodes at the thrombus includes radially expanding the expandable member.

19. The method of claim 16, wherein treating the fibrin of the thrombus after the PFA energy is delivered to the red blood cells of the thrombus comprises aspirating the fibrin from the vessel.

20. The method of claim 16, wherein treating the fibrin of the thrombus after the PFA energy is delivered to the red blood cells of the thrombus comprises applying a thrombolytic drug to the fibrin to lyse the fibrin.
